(19) United States
(12) Patent Application Publication
(10) Pub. No.: US 2014/0373831 A1
(43) Pub. Date: Dec. 25, 2014

(54) PRE-FILLED DISPOSABLE NEBULIZER CHAMBER

(71) Applicants: Andrew R. CULBERTSON, Manchester, OH (US); Richard A. GENGA, JR., East Greenwich, RI (US)

(72) Inventors: Andrew R. CULBERTSON, Manchester, OH (US); Richard A. GENGA, JR., East Greenwich, RI (US)

(21) Appl. No.: 14/309,333

(22) Filed: Jun. 19, 2014

Related U.S. Application Data
(60) Provisional application No. 61/836,728, filed on Jun. 19, 2013.

(51) Int. Cl. A61M 11/00; B65B 3/04; B65B 7/16; A61J 7/00

(52) U.S. Cl. CPC A61M 11/00; A61J 7/0053; B65B 3/04; B65B 7/16; A61M 2207/00
USPC 128/200.21; 206/438; 53/467

(57) ABSTRACT

A pre-filled, single-use, disposable medication chamber for use in a medical nebulizer system that delivers a mist of properly sized aerosol particles of medicament to the patient. The nebulizer can be effectively used in conjunction with conventional nebulizer cap, tee and mouthpiece devices that interface with the patient.

### CROSS-REFERENCE TO RELATED APPLICATIONS

This application claims the benefit of U.S. Provisional Application 61/836,728 filed Jun. 19, 2013, the disclosure of which is incorporated herein by reference.

### FIELD OF THE INVENTION

The present invention relates generally to inhalation devices, and more particularly, to a pre-filled, single-use, disposable medicinal chamber for patient nebulizer systems.

### BACKGROUND OF THE INVENTION

In medicine, a nebulizer is defined as a device that is used to administer medication to the patient's airways in the form of a liquid mist, more properly known as an aerosol. In general the prior art devices used for producing medical aerosols fall into two categories; the small volume nebulizer (SVN), and the metered dose inhaler (MDI). The small volume nebulizer (SVN) has traditionally been the apparatus of choice for delivery of therapeutic aerosols. The delivery apparatus typically consists of a multi-use disposable or reusable nebulizer, a mouthpiece or facemask, and a pressurized gas source usually oxygen or air. The metered dose inhaler (MDI), on the other hand, typically contains the active drug, dissolved in chlorofluorcarbon (CFC) or chlorofluroalkane (CFA) propellants and excipients plus a metering valve. The drug-containing canister of the device is generally fitted to a mouthpiece actuator and spacer or valved holding chamber, and activation of the device by compressing it results in the release of a metered dose of medication.

FIG. 1 (a perspective assembled view) and FIG. 2 (an exploded view) show a conventional nebulizer unit useful to deliver liquid medication to a patient in an aerosol form. These devices are not life-supporting or life-sustaining, nor implanted. The devices are typically for single patient use. The nebulizer system 1 device is typically comprised of the following components. A tee (T-shaped tube piece) 2 has one leg of the tee attached to a capture reservoir 3 and the other leg connected to a mouth piece 4. The third upright leg of the tee is connected to the top outlet port 5a of a nebulizer top cap unit 5. A base chamber 7 has a top opening defined by a threaded rim 7a that threads into the mating threaded skirt 5b of the nebulizer top cap unit 5. A venturi cone 6 is disposed within the cavity of the base chamber 7, with a conical wall 6a tapering from a wide rim 6b to a tapered port 6c. The tapered wall 6a of the venturi cone 6 is positioned in registry with a conical wall 7c within the base chamber 7 to define a conical conduit therebetween, in liquid communication with an annular floor 7d. The base chamber 7 also includes an axial inlet port 7b which passes upward and through the apex of the conical wall 7c to form an axial outlet port 7e.

FIGS. 3, 4 and 5 show the conventional use of the nebulizer devices in a system to deliver liquid medication to a patient in an aerosol form. FIG. 3 shows a unit-dose medicament package 10 that includes a container 10a filled with a single dose of a liquid medicament (LM) and a tear-away closure 10b that forms a dispensing port 10c when torn away. The base chamber 7 is unscrewed or detached from the nebulizer top cap 5, and the LM from the single-dose package is expressed into the base chamber 7 to flood the annular floor 7d. The venturi cone 6 is then inserted into the opening of the base chamber 7, which is then screwed onto or reattached to the nebulizer top cap unit 5. The conical conduit space 11 (FIG. 5) is formed between the wall 6a of the venturi cone 6 and the wall 7c of the base chamber 7, and provides liquid communication from the pool of liquid medicament LM along the floor 7d to the tapered port 6c of the venturi cone 6, as shown in FIG. 5. Air flows up into the inlet port 7b and out of the outlet port 7e. The stream of air draws liquid medicament LM from within the conical conduit space 11 into the air stream exiting the tapered port 6c of the venturi cone 6. The droplets of liquid carried in the air stream impinge upon a deflector plate 12 to form the aerosol or mist. Liquid entrainment is further enhanced by directing the combined liquid and gas flow past a series of radial deflectors causing turbulence in the stream. The combination of these design features results in generation of a very fine FDA specified range of aerosolized liquid droplets 25 in the aerosol-gas mixture that then flows out of the top outlet port 5a of a nebulizer top cap unit 5, and into third upright leg 2c of the tee 2, for delivery to the patient.

Various types of prior art inhalers have also been offered for sale and are in wide use. Inhalers have the advantage of portability but have been criticized on the basis that patients often lack the coordination and psychomotor skills to use them properly without professional supervision. This dichotomy of available device types (nebulizers vs. inhalers) has lead to a great deal of controversy regarding which method is superior, although many experts have concluded that nebulizers and inhalers are essentially equivalent in terms of therapeutic outcomes. Accordingly, in many respects, the choice of device revolves around non-outcome related factors such as cost, convenience, ease-of-use, safety, patient preference, patient compliance and adherence, as well as the availability of medications in one or both delivery forms. Despite alternative methodologies, it is clear that inhaled medication delivery by nebulizer is a permanent part of the treatment options for respiratory disease patients and is becoming a useful tool for systemic drug delivery as well.

This being the case, there is an abundance of plastic disposable medication nebulizers on the market, but the vast majority of these devices are essentially clones, differing from one another mainly in appearance. Functionally, they are essentially identical. The overriding similarity between all these devices is that they are all supplied empty and the medication they are to nebulize must be transferred into them prior to commencement of the treatment by either the professional respiratory therapist in the hospital setting or the patient or patient's caregiver in the home setting.

Recently, various investigators and companies have sought to improve liquid nebulization. However, due to the physics of jet nebulization, the possibility of performance improvements in the jet nebulizer itself are very limited. Many of the improvements have involved electronic controlled or driven nebulizers that have improved efficiency but are also so expensive as to be out-of-reach for the typical routine nebulization purposes.

As will be discussed more fully hereinafter, there are various well recognized technical limitations to nebulizer use. These include the following:
1. Excessive patient dosing time.
2. Dose of drug delivered to the patient is undesirably affected by patient breathing parameters that may result in unacceptable variations in drug delivery dose.
3. Cleaning of the nebulizer after each use is time-consuming and frequently neglected thereby providing a possible avenue for nosocomial infection (bacteria/viral spread within a healthcare organization).
4. In a hospital environment, excessive amounts of technologist time is required for each patient treatment.
5. Release of the drug to atmosphere is not only wasteful but can be detrimental to healthcare workers who breathe “second-hand” aerosol drugs.
6. Because of lengthy treatment times, patients may become fatigued and compliance is compromised.
7. Hospital use is often determined by price only, not performance.

In light of the aforementioned drawbacks, Dr. J. H. Dennis, a highly recognized aerosol researcher, has stated as follows in the Practical Handbook of Nebulizer Therapy. London, Martin Dunitz; 2004: 42-43: “It is clear that neither pressurized metered dose MDI's, nor DPI's meet all the necessary requirements despite the enormous amounts of pharmaceutical funding which has been devoted to improvement of these devices over the past three decades.”

The present invention addresses this problem by providing a sealed, pre-filled, disposable, small volume medicinal chamber of the invention for use with a nebulizer system.

### SUMMARY OF THE INVENTION

An aspect of the present invention is a sealed, pre-filled, disposable medicinal chamber, which includes a medicinal base chamber, a venturi cone, a pre-determined dose quantity of a liquid medicament, and a means for sealing the liquid medicament within the base chamber from the outside environment, for use with a nebulizer system that delivers a mist of properly sized aerosol particles of medicament to the patient with a very high-level of efficiency.

Another aspect of the invention is to provide a medicinal chamber of the aforementioned character that comprises packaging an inhalation drug in a preferred unit-dose, disposable container that confers the benefits of unit-dose packaging while it simultaneously performs the function of highly effective drug aerosolization.

Another aspect of the invention is to provide a medicinal chamber of the character described that is small in physical size for convenience of packaging, storage, dispensing and operation.

Another aspect of the invention is to provide a pre-filled, disposable, medicinal chamber that interfaces with a conventional nebulizer cap to deliver a mist of properly sized aerosol particles of medicament to the patient with a very high-level of efficiency.

A further aspect of the invention is to provide a pre-filled, disposable, medicinal chamber that can be produced in large quantities at minimal cost by conventional thermoplastic injection molding means and medicinal filling and handling technologies.

Another aspect of the invention is to provide a sealed, pre-filled, disposable, medicinal chamber as described in the preceding paragraphs that can be effectively used in conjunction with conventional nebulizer cap, tee and mouthpiece patient interface devices, as well as with more sophisticated patient interface devices such as dosimetric/reservoir systems, or mechanical ventilator systems.

Another aspect of the invention is to provide a pre-filled, disposable medicinal chamber that effectively mitigates against the possibility of self-contamination or cross-contamination due to improper cleaning of the device.

Another aspect of the invention is to provide a disposable medicinal chamber of the class described that effectively minimizes practitioner set-up and preparation time thereby conferring significant labor savings benefits upon healthcare organizations that employ such practitioners for the purpose of administering medicated aerosol therapy.

Another aspect of the invention is to provide a sealed, pre-filled, disposable medicinal chamber, which includes a medicinal base chamber, a venturi cone, a pre-determined dose quantity of a liquid medicament, and a means for sealing the liquid medicament within the base chamber from the outside environment, that effectively reduces or eliminates practitioner clean-up time following administration of the contained medication, thereby conferring significant labor savings benefits upon healthcare organizations that employ such practitioners for the purpose of administering medicated aerosol therapy.

Another aspect of the invention is an article of manufacture comprising a sealed, pre-filled, disposable medicinal chamber.

Another aspect of the invention is a method of preparing a sealed, pre-filled, disposable medicinal chamber, comprising the steps of: a) providing disposable medicinal chamber according to claim 1; b) pre-filling a dose amount of a liquid medicine into an annular trough of the disposable medicinal chamber; c) applying a film seal to a top opening of the medicinal chamber, and d) applying a seal closure to an inlet air port of the medicinal chamber.

Another aspect of the invention is a method for improving patient compliance in administering medicated aerosol therapy, comprising the steps of: a) providing a sealed, pre-filled, disposable medicinal chamber containing a pre-filled dose amount of a liquid medicine; b) removing a top opening film seal from the top opening of the medicinal chamber; c) attaching and sealing the top opening of the pre-filled base chamber to a nebulizer top cap unit; d) removing an inlet air port seal from the inlet air port of the medicinal chamber, e) connecting tubing of a pressurized air pump supply tubing to the inlet air port. The assembled nebulizer unit is then used for administering the aerosol medication to the patient.

### BRIEF DESCRIPTION OF THE DRAWINGS

FIG. 1 is a perspective view of an assembled, conventional nebulizer system.

FIG. 2 is an exploded view of the components of the conventional nebulizer system of FIG. 1.

FIG. 3 is a disposable, conventional container of a liquid medicament.

FIG. 4 is a schematic view of the assembled, conventional nebulizer system being used to delivers a mist of aerosol particles of the liquid medicament to a patient.

FIG. 5 is a detailed view of the conventional nebulizer system of FIG. 4 showing the formation of the mist of aerosol particles of the liquid medicament.

FIG. 6 is a perspective view of a sealed, pre-filled, disposable medicinal chamber of the present invention.

FIG. 7 is an exploded view of the components of the sealed, pre-filled, disposable medicinal chamber of FIG. 6.

FIG. 8 is a cross sectional view of the sealed, pre-filled, disposable medicinal chamber, viewed through line 8-8 of FIG. 6.

FIG. 9 is an exploded, cross sectional view of the components of the sealed, pre-filled, disposable medicinal chamber, viewed through line 9-9 of FIG. 7.

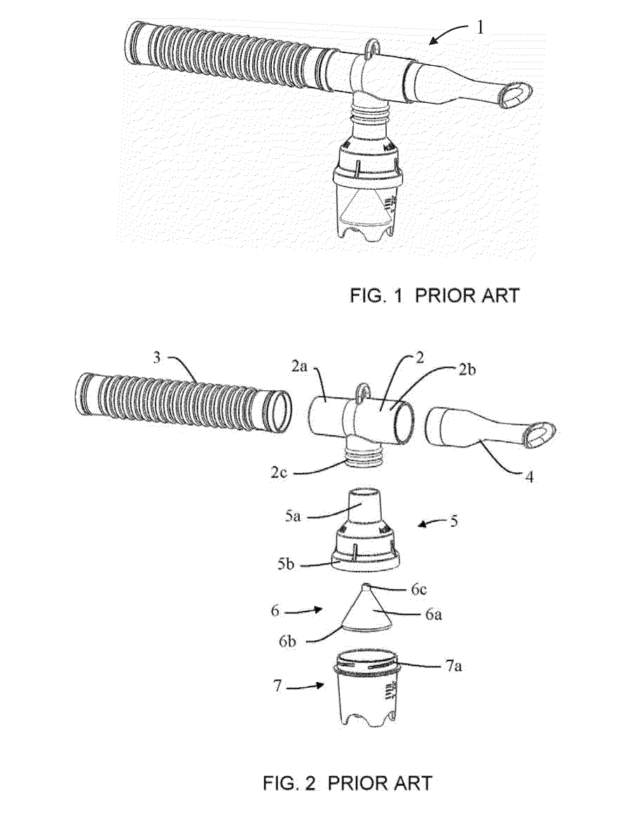

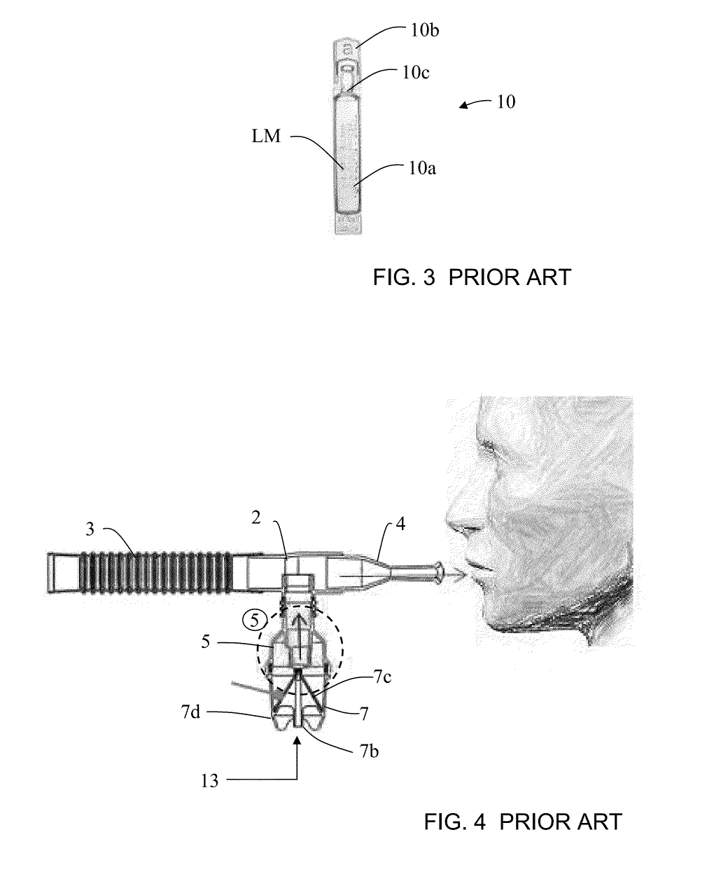

### DETAILED DESCRIPTION OF THE INVENTION

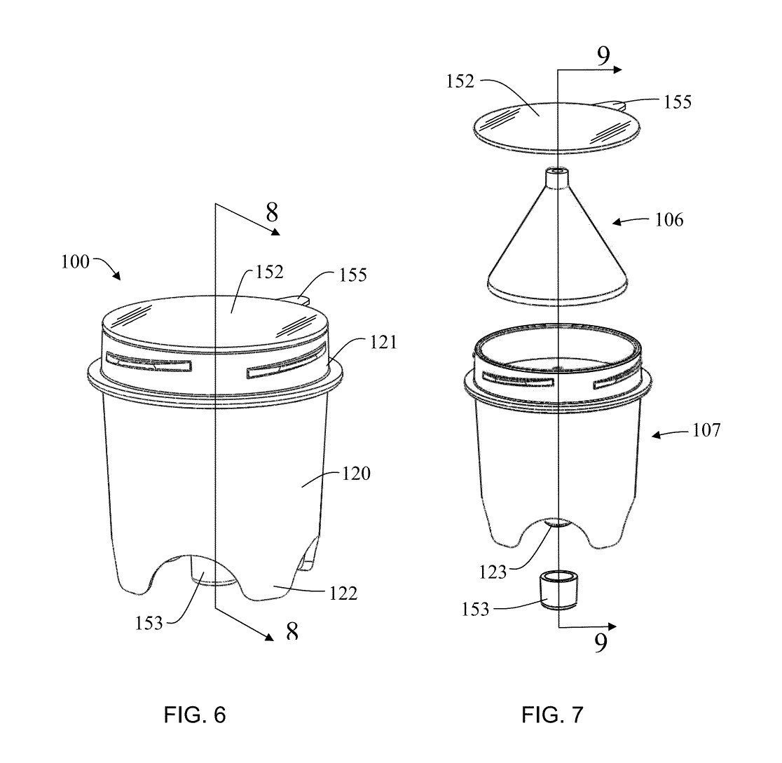

Referring to the drawings, and particularly to FIGS. 6 through 9, an embodiment is shown of a sealed, pre-filled, disposable medicinal chamber 100 of the invention for delivering a multiplicity of particles of aerosolized medication of a selected size to a patient. The sealed medicinal chamber 100 is supplied pre-filled with the required inhalable dose(s) of liquid medication (LM), used for a single treatment (or for two or more treatments), and then discarded or disposed.

The sealed medicinal chamber 100 includes a venturi cone 106 and a base chamber 107. Sealing means for sealing the medicinal chamber include a film seal 152 for the top opening of the base chamber, and a seal closure 153 for the an inlet air port of the base chamber. The film seal 152 provides a vapor-proof barrier, including a vapor- and liquid-proof barrier, and can include a metallic foil layer. The metallic foil layer can include an aluminum foil layer. The film seal can also include a polymeric inner heat seal layer 154, for removably sealing and bonding the film seal 152 to the upper edge of the threaded rim 121 of the base chamber. The seal closure 153 can include a cap that fits over the outer wall of the inlet air port, including a frictional fit that retains the cap over the outer wall of inlet air port until manually pried off. The seal closure 153 is typically a silicone or rubber material that fits snuggly, though removably, to the inlet port, and which provides a vapor and liquid seal to the inlet air port 123. Alternatively, seal closure for the inlet air port can be a plug that fits within the inlet air port 123, including a frictional fit that retains the plug within the inlet air port until manually extracted. The sealing means prevent leakage of the liquid medicament LM or its vaporous components from escaping the sealed medicinal chamber 100 after assembly, during shipment and storage, until removed for use.

The base chamber 107 provides a vessel for containing the dose(s) of liquid medicament LM. The base chamber 107 also provides a means for delivering liquid that mixes with supplied pressurized air in a venturi mixing device, described below. The base chamber 107 includes an outer container wall 120 that extends upward to a threaded rim 121 that defines an upper opening 129. The threaded rim 121 provides a means for threadedly and sealingly connecting the base chamber 107 to a conventional nebulizer unit, shown as nebulizer top cap unit 5 in FIG. 2. The body of the base chamber 107 can includes a plurality of legs 122 for placing and stabilizing the base chamber 107 on a flat support surface during storage, assembly and use.

As shown in detail in FIG. 9, the base chamber 107 also includes an internal wall structure 124 having a conical outer surface 125 that defines an annular trough 126 with the outer wall 120. An airflow passage 128 extends from the inlet air port 123, through the internal wall structure 124, to an axial outlet port 127.

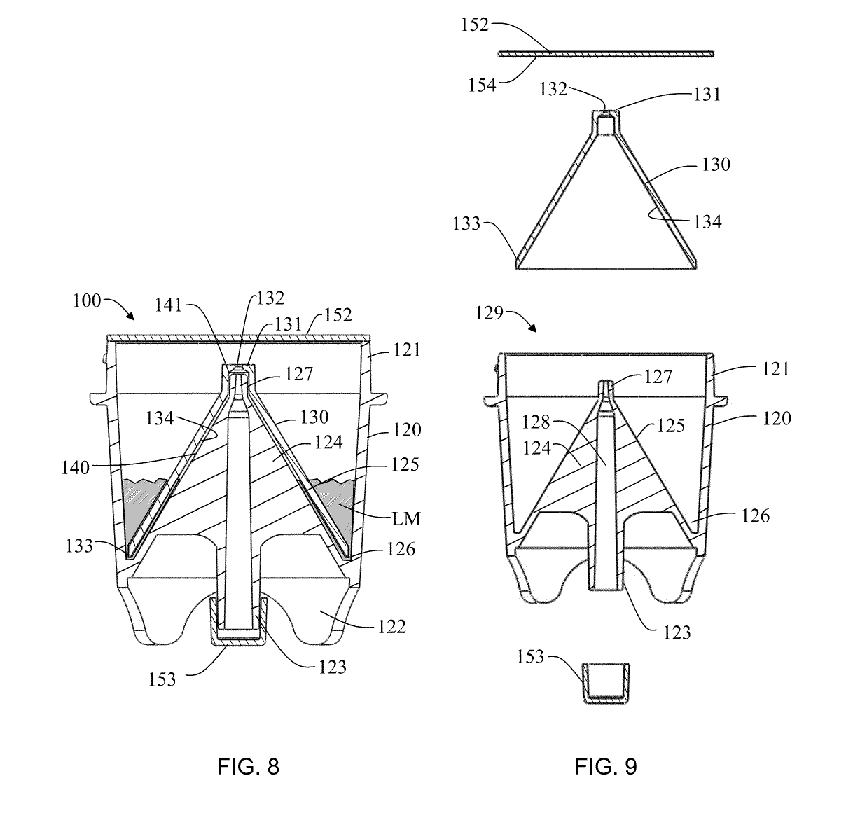

The venturi cone 106 includes a conical wall 130 having an inner surface 134, a tapered end 131 having an exit opening 132, and an annular rim 133. The inner surface 134 of the conical wall 130 is configured to confront the conical outer surface 125 of the base chamber 107 to define a conical conduit 140 (FIG. 8) therebetween. The conical conduit 140 provides liquid communication with the annular trough 126 of the base chamber 107. The tapered end 131 has a cylindrical member extending from the end of the conical wall 130 to an outer wall that has the exit opening 132. The inner surface of the tapered end 131 is configured to confront the outer surface of the outer port 127 of the base chamber to define a discharge conduit 141 therebetween in liquid communication with the conical conduit 140. The distal end of the discharge conduit 141 communicates with the passage 128 at the outlet port 127.

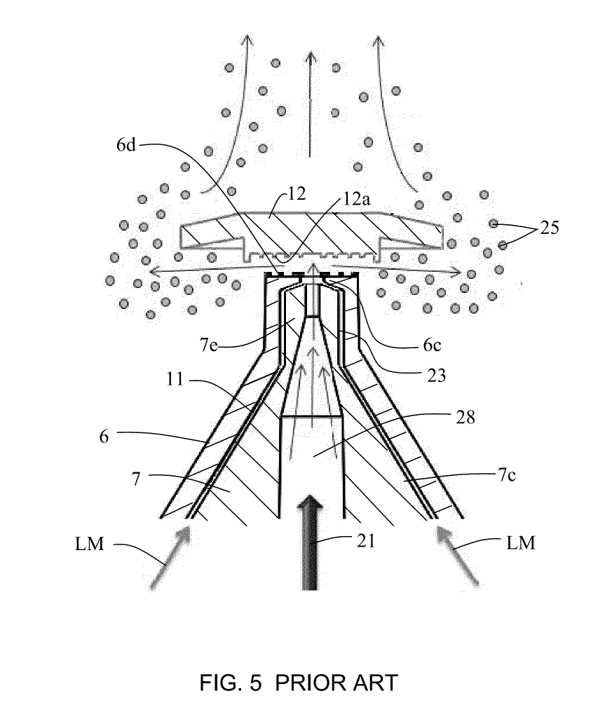

The inlet air port 123 is configured for connection to a conventional supply of pressurized air through flexible tubing 13 (FIG. 4). As illustrated in a conventional nebulizing system in FIG. 5, when the pressurized air 21 is passed through the passage 28, the rapid flow of air through the outlet port 6c draws liquid medicament LM via the venturi effect from the conical conduit 11 between the confronting conical surfaces, and through the discharge conduit 23. The amount of the liquid medicament drawn from the discharge conduit 23 that is entrained in the air stream is enhanced by directing the flow of liquid and gas against a deflector member 12 in the nebulizer unit. Liquid entrainment is further enhanced by directing the combined liquid and gas flow past a series of radial deflectors 12a in the deflector member 12 and/or radial deflectors 12b on the outlet port 6c, or other features that can cause turbulence in the air stream, including right angle turns of the air flow. The combination of these design features results in generation of a very fine FDA-specified range of aerosolized liquid and gas mixture for delivery to the patient.

Similarly in the present invention, in the illustrated device in FIGS. 8 and 9, when the pressurized air is passed through the passage 128, the rapid flow of air through passage of the outlet port 127 draws liquid medicament LM, that has accumulated in the annular trough 126, through the conical conduit 140 and the discharge conduit 141. The flow of air is typically created by an electrically powered air pump that generates approximately 12 to 25 psi of pressurized air at a flow rate of approximately 4 to 8 liters per minute. The flow of air during the treatment is continued until all the liquid medicament has been drawn out of the annular trough 126 and through the conical conduit 140 and discharge conduit 141.

Examples of chamber bases, venturi cones, and nebulizer units are disclosed, for example, in U.S. Pat. No. 4,746,067, RE 33,717, and U.S. Application Publication 2010/0095958, the disclosures of which are incorporated by reference in their entireties.

The sealed medicinal chamber 100 is assembled by installing the venturi cone 106 into the base chamber 107, dispensing a pre-determined dose quantity of the liquid medicament into the base chamber 107, and then sealing both the top opening 129 of the base chamber 107 and the inlet air port 123. Sealing means include a top opening film seal 152 and an inlet air port seal (airway plug) 153.

Examples of the liquid medicament LM that can be employed in the invention include, but are not limited to, liquid bronchodilator medications:, including albuterol sulfate inhalation solution, ipratropium bromide inhalation solution, cromolyn sodium inhalation suspension, budesonide inhalation suspension, pirbuterol, anti-IgE, arformoterol, and formoterol.

In a method of the present invention, the top opening seal 152 of the sealed, pre-filled, disposable medicinal chamber is peeled off by the patient or medical personnel. The top opening seal 152 can include a tab 155 or similar removal aid means extending from a peripheral edge to facilitate manual peeling and removal of the film seal 152 up and off of the upper edge of the rim 121. The base chamber is threaded and sealed to a nebulizer top cap unit. The inlet air port seal is then removed from the inlet air port, and the supply tubing of a pressurized air pump is connected to the inlet air port. The nebulizer is then employed conventionally. The method provides improved patient compliance in the administration of aerosol medication, for patients who may be fatigued, that helps to avoid over- or under-mediating due to mistakes in measuring the correct dosage, and avoids the time needed for measure and dosing the medicament at a time when the patient may be having difficulty breathing.

The present invention involves liquid medication packaging that is an integral functional component of a jet nebulizer, and combines, integrates, and seals nebulizer liquid medication into one of the functional components of the jet nebulizer (in this case, the base chamber, such that this functional component now becomes the means for packaging the liquid medicament. The integration could include single dose or multi-dose capabilities.

The bronchodilator or other liquid medication is packaged and stored within the device in a manner that eliminates the need of the user to properly clean, measure, and fill the device correctly before each use, as presently practiced. The conventional medical atomizing devices require the user or the medical caretaker to properly open and fill the medicine reservoir transferring it manually from medicine container to the device. Great care to measure the correct dosage and more importantly critical time is lost during the transfer, especially when the user is self-administrating the treatment while having difficulty breathing. In many cases of asthmatic onset or COPD illness the user is confused from the effects caused by not being able to properly breathe. Having to assemble the device and transfer the medication has been identified as a cause in a critical delay of treatment. At the onset of breathing difficulty the results commonly creates additional medical complications known to be produced from hypoxia that can cause the patient to pass out and or be further injured before accomplishing the task of assembly and treatment.

The present invention and its approach not only reduces the time to start treatment but it increases the quality of care by eliminating mistakes in measuring the proper dose of medication, spilling during the transfer exposing medication to others and pets, contamination from open exposure in the home setting during the manual transfer of medication to the device which is commonly done at the kitchen table or food eating areas. This device can be used as a one-time use eliminating the need to properly clean and store for next use which is a common cause of the device malfunctioning at a critical moment of need requiring the device to be opened cleaned to remove the blockage and refilled with medication.

The cost of a disposable jet style nebulizer is typically less than or equal to the cost of the packaging of the medication that is used with a nebulizer. Combining the liquid medication and packaging into a sealed integral functional part of the nebulizer provides a user with higher quality medical care while also generating a market advantage via elimination of waste and simplification of manufacturing logistics. The present invention can:
a) Streamline/reduce the current parallel logistical and manufacturing redundancies via reduction and elimination of materials and upstream labor to produce the millions of disposable plastic medication vials used annually.
b) Improve patient quality of care.
c) Reduce non-sterile human contact/contamination via pre-sealed integral packaging.
d) Reduce required amounts of medication: Current vial designs require manual transfer of liquid medication from the vial to the device medication reservoir. Additional medication is needed to compensate for the transfer loss that exists during the transfer.
e) Reduce the spill risk associated with manual transfer of the medication to the nebulizer, thus reducing potential exposure to children and pets of spilled medications in the home care environment.

Having now described the invention in detail in accordance with the requirements of the patent statues, those skilled in this art will have no difficulty in making changes and modifications in the individual parts or their relative assembly in order to meet specific requirements or conditions. Such changes and modifications may be made without departing from the scope and spirit of the invention, as set forth in the following claims:

1. A sealed, pre-filled, disposable medicinal chamber, which includes a medicinal base chamber for containing a pre-determined dose quantity of a liquid medicament, a venturi cone, and a means for sealing the liquid medicament within the base chamber from the outside environment, for use with a nebulizer system that delivers a mist of properly sized aerosol particles of medicament to the patient with a very high-level of efficiency.

2. The sealed, pre-filled, disposable medicinal chamber according to claim 1, wherein the medicinal base chamber and a venturi cone are made of a thermoplastic material.

3. The sealed, pre-filled, disposable medicinal chamber according to claim 1, wherein the medicinal base chamber has an annular sidewall defining a top opening, and the sealing means includes a vapor-proof film sealed along the top opening of the annular sidewall.

4. The sealed, pre-filled, disposable medicinal chamber according to claim 3, wherein the medicinal base chamber includes an inlet port, and the sealing means includes a plug removable disposed over the inlet port.

5. The sealed, pre-filled, disposable medicinal chamber according to claim 1, wherein the medicinal base chamber includes an inlet port, and the sealing means includes a seal plug removably disposed over the inlet port.

6. The sealed, pre-filled, disposable medicinal chamber according to claim 1, wherein the liquid medicament is a bronchodilator.

7. An article of manufacture comprising a sealed, pre-filled, disposable medicinal chamber according to claim 1, and a pre-determined dose quantity of a liquid medicament contained within the medicinal base chamber.

8. A method of preparing a sealed, pre-filled, disposable medicinal chamber, comprising the steps of: a) providing disposable medicinal chamber according to claim 1; b) pre-filling a dose amount of a liquid medicine into an annular trough of the disposable medicinal chamber; c) applying a film seal to a top opening of the medicinal chamber, and d) applying a seal closure to an inlet air port of the medicinal chamber.

9. A method for improving patient compliance in administering medicated aerosol therapy, comprising the steps of: a) providing a sealed, pre-filled, disposable medicinal chamber containing a pre-filled dose amount of a liquid medicine; b) removing a top opening film seal from the top opening of the medicinal chamber; c) attaching and sealing the top opening of the pre-filled base chamber to a nebulizer top cap unit; d) removing an inlet air port seal from the inlet air port of the medicinal chamber, e) connecting tubing of a pressurized air pump supply tubing to the inlet air port.
